(19) United States
(12) Patent Application Publication
(10) Pub. No.: US 2022/0409517 A1
(43) Pub. Date: Dec. 29, 2022

(54) TRANSPARENT COMPOSITION FOR USE IN SKINCARE AND/OR HAIRCARE

(71) Applicant: S.P.C.M. SA, ANDREZIEUX-BOUTHEON (FR)

(72) Inventors: Xavier ALVES, ANDREZIEUX CEDEX (FR); Frédéric BLONDEL, ANDREZIEUX CEDEX (FR)

(21) Appl. No.: 17/787,612

(22) PCT Filed: Dec. 22, 2020
(86) PCT No.: PCT/EP2020/087695

§ 371 Date: Jun. 21, 2022

(30) Foreign Application Priority Data
Dec. 27, 2019 (FR) 19 15668

(51) Int. Cl. A61K 8/81; A61Q 5/02; A61Q 19/00; C08F 220/60; C08F 220/34; C08F 220/58

(52) U.S. Cl. CPC A61K 8/8188; A61Q 5/02; A61Q 19/00; C08F 220/60; C08F 220/34; C08F 220/585; A61K 2800/262; A61K 2800/5428; C08F 2800/10

(57) ABSTRACT

This invention concerns a transparent composition comprising at least a first amphoteric water-soluble polymer, which comprises:
A units of a cationic monomer selected from: acidified or quaternised dimethylaminoethyl acrylate salts, acidified or quaternised dimethylaminoethyl methacrylate salts, acidified or quaternised dimethylaminopropyl acrylamide salts, and acidified or quaternised dimethylaminopropyl methacrylamide salts, and mixtures thereof;
B units of 2-acrylamido-2-methylpropanesulphonic acid and/or salts thereof; and
optionally C units of acrylamide,

characterised in that the molar ratio between the A units of cationic monomer and the B units of 2-acrylamido-2-methylpropanesulphonic acid and/or salts thereof is between 0.4 and 1.4.

### FIELD OF THE INVENTION

The invention concerns an amphoteric water-soluble polymer. The invention also concerns a composition comprising an amphoteric water-soluble polymer and, in particular, a haircare and/or skincare composition comprising an amphoteric water-soluble polymer. The invention further concerns the use of the amphoteric water-soluble polymer as a conditioning agent in a haircare and/or skincare composition.

### PRIOR ART

Most shampoos on the market are ‘2-in-1’ shampoos that serve to wash hair, but also soften it. In order to fulfill the first function, surfactants are used, particularly anionic and/or zwitterionic surfactants, which ensure that the shampoo is not too harsh on the hair and scalp. Softness, on the other hand, is provided by conditioning agents, which are silicone agents, i.e. polymers having cationic charges with an affinity for hair. The greater the cationic charge of this polymer, the better this affinity with the keratinous fibres of hair.

The combination of the two agents is frequently used, with the cationic polymer allowing for better deposition of the silicone agent on the hair by means of a coacervation mechanism. Coacervation results from the ionic attraction between two compounds having opposite charges. In particular, coacervates may result from the ionic attraction between anionic surfactants and a cationic polymer.

It is known that these coacervates have interesting conditioning and active agent deposition aiding properties (Principles of polymer science and technology in cosmetics and personal care—Polymer/surfactant interaction in applied systems E.D Goddard).

However, the use of anionic surfactants and a polymer with cationic charges in a single shampoo composition results in significant incompatibilities. This translates into an opaque appearance of the composition, which is not desirable to the formulator or user. There, too, the greater the cationic charge of the polymer, the greater these incompatibilities are.

Moreover, conditioning agents such as silicone agents are substances with substantial costs in a shampooing composition, the deposition of which on the hair is rarely optimised, as they are carried by the surfactants during the rinsing stage.

Document US 2003/0022987 describes a polymer containing a quaternised monomer, a crosslinking agent, and, optionally, an anionic monomer and a non-ionic agent. This polymer is used in fields in which deposition is important, such as paper manufacturing, inkjet printing, cement manufacturing, drilling of wells or subterranean formations, or in cosmetic or shampoo compositions. The use of a crosslinking agent may result in a gel that is too viscous and incompatible with use in haircare and/or skincare formulations.

Document U.S. Pat. No. 5,879,670 describes a method for treating keratin comprising contacting the keratin with a cosmetic composition comprising an amphoteric polymer. The polymer in question provides an improvement of the conditioning properties of the cosmetic composition. However, the polymer described in this document does not offer satisfactory properties in terms of the transparence of the formulation.

Document US 2008/0199416 describes a method for modifying the rheology of a cosmetic composition for hair using an amphoteric polymer and a crosslinking agent. The amphoteric polymer makes it possible for a cosmetic composition to take the form of a clear gel, and to improve the conditioning properties of the composition. The use of a crosslinking agent may cause numerous problems, in particular excessive viscosity of the gel.

Despite these technological developments, there is a need to improve the properties of haircare and/or skincare compositions in order to obtain clear compositions comprising highly charged cationic polymers that improve haircare and/or skin affinity whilst maintaining good active agent deposition properties.

This invention solves these issues by proposing a composition comprising a highly cationic amphoteric water-soluble polymer. In the presence of anionic surfactants, the amphoteric water-soluble polymer allows for a transparent composition to be obtained, and for increased active agent deposition due to the coarcervation mechanism, even when the amphoteric water-soluble polymer is highly cationic.

The amphoteric water-soluble polymer has a better affinity with haircare and/or skin, and acts as a deposition aid when included in a cosmetic composition.

Moreover, the amphoteric water-soluble polymer also provides improved performance in the field of household detergents, where their deposition-aiding action allows for easier active agent deposition on negatively charged surfaces.

### DISCLOSURE OF THE INVENTION

This invention concerns a composition comprising at least one amphoteric water-soluble polymer, which comprises:
A units of a cationic monomer selected from: acidified or quaternised dimethylaminoethyl acrylate salts, acidified or quaternised dimethylaminoethyl methacrylate salts, acidified or quaternised dimethylaminopropyl acrylamide salts, and acidified or quaternised dimethylaminopropyl methacrylamide salts, and mixtures thereof;
B units of 2-acrylamido-2-methylpropanesulphonic acid (ATBS) and/or salts thereof; and
optionally C units of acrylamide,
   characterised in that the molar ratio between the A units of cationic monomer and the B units of 2-acrylamido-2-methylpropanesulphonic acid and/or salts thereof is between 0.4 and 1.5.

Another object of the invention concerns an amphoteric water-soluble polymer, comprising:
A units of a cationic monomer selected from: acidified or quaternised dimethylaminoethyl acrylate salts, acidified or quaternised dimethylaminoethyl methacrylate salts, acidified or quaternised dimethylaminopropyl acrylamide salts, and acidified or quaternised dimethylaminopropyl methacrylamide salts, and mixtures thereof;
B units of 2-acrylamido-2-methylpropanesulphonic acid; and
optionally C units of acrylamide,
   characterised in that the molar ratio between the A units of cationic monomer and the B units of 2-acrylamido-2-methylpropanesulphonic acid and/or salts thereof is between 0.4 and 1.5.

Another object of the invention concerns the use of the amphoteric water-soluble polymer according to the invention in cosmetics, in particular to improve the active agent deposition on haircare and/or skin in a haircare and/or skincare composition.

Another object of the invention concerns the use of the amphoteric water-soluble polymer according to the invention to improve active agent deposition in a detergent composition.

Another object of the invention concerns the use of the amphoteric water-soluble polymer according to the invention to improve active agent deposition in a household fabric softener composition.

### Amphoteric Water-Soluble Polymer According to the Invention

The amphoteric water-soluble polymer according to the invention comprises:
A units of a cationic monomer selected from: acidified or quaternised dimethylaminoethyl acrylate salts, acidified or quaternised dimethylaminoethyl methacrylate salts, acidified or quaternised dimethylaminopropyl acrylamide salts, and acidified or quaternised dimethylaminopropyl methacrylamide salts, and mixtures thereof;
B units of 2-acrylamido-2-methylpropanesulphonic acid (ATBS) and/or salts thereof;
optionally C units of acrylamide,

and is characterised in that the molar ratio between the A units of cationic monomer and the B units of 2-acrylamido-2-methylpropanesulphonic acid and/or salts thereof is between 0.4 and 1.5.

‘X units’ of a monomeric composition refers to X units resulting from the polymerisation of the monomeric composition.

‘Water-soluble polymer’ refers to a polymer that provides an aqueous solution when dissolved with stirring at 25° C. and a concentration of 10 g·L⁻¹ in water.

Acidified salts may, inter alia, be obtained by protonation. Quaternised salts may be obtained by reaction with benzyl chloride, methyl chloride (MeCl), aryl and alkyl chlorides, or dialkylsulphates such as dimethylsulphate. Preferably, the quaternised salts are chloride salts.

In a particular embodiment of the invention, the amphoteric water-soluble polymer is a copolymer of quaternised dimethylaminoethyl acrylate (DMAEA), 2-acrylamido-2-methylpropanesulphonic acid (AMPSA), and/or salts thereof, and optionally acrylamide.

In a particular embodiment of the invention, the amphoteric water-soluble polymer is a copolymer of quaternised dimethylaminoethyl methacrylate (DMAEMA), 2-acrylamido-2-methylpropanesulphonic acid (AMPSA), and/or salts thereof, and optionally acrylamide.

In a particular embodiment of the invention, the amphoteric water-soluble polymer is a copolymer of acrylamido propyltrimethyl ammonium chloride (APTAC), 2-acrylamido-2-methylpropanesulphonic acid (AMPSA), and/or salts thereof, and optionally acrylamide.

In a preferred embodiment of the invention, the amphoteric water-soluble polymer is a copolymer of methacrylamido propyltrimethyl ammonium chloride (MAPTAC), 2-acrylamido-2-methylpropanesulphonic acid (AMPSA), and/or salts thereof, and optionally acrylamide.

Preferably, the amphoteric water-soluble polymer according to the invention comprises a high cationic charge. ‘High cationic charge’ means that it comprises at least 30 mol % of A units of cationic monomer. Above this value, fixation of the amphoteric water-soluble polymer on the hair becomes more difficult, and the conditioning effect is reduced.

The quantity of A units of cationic monomer in the amphoteric water-soluble polymer according to the invention is preferably greater than or equal to 30 mol relative to the molar quantity of all units included in the polymer. It is preferably between 30 and 60 mol %. More preferably, it is between 40 and 55 mol %.

The quantity of B units of 2-acrylamido-2-methylpropanesulphonic acid (AMPSA) in the amphoteric water-soluble polymer according to the invention is preferably between 20 and 70 mol % relative to the molar quantity of all units included in the polymer. It is preferably between 25 and 60 mol %. More preferably, it is between 30 and 50 mol %.

The sum of the monomers, in particular A, B, and, optionally, C units, can be adjusted by persons skilled in the art to be equal to 100 mol % in the amphoteric water-soluble polymer according to the invention.

The B units of 2-acrylamido-2-methylpropanesulphonic acid may be salified in whole or in part. The salified form corresponds advantageously to alkaline metal salts (Li, Na, K, etc.), alkaline earth metals (Ca, Mg, etc.), or ammonium salts, in particular quaternary ammonium salts. Preferred are sodium salts.

Preferably, the B units of 2-acrylamido-2-methylpropanesulphonic acid are partially salified, preferably between 50 and 100% relative of the total weight of the B units of 2-acrylamido-2-methylpropanesulphonic acid are salified.

Preferably, the molar ratio between the A units of cationic monomer and the B units of 2-acrylamido-2-methylpropanesulphonic acid is between 0.4 and 1.5, preferably between 0.4 and 1.4, preferably between 0.4 and 1.35, more preferably between 0.6 and 1.3.

Preferably, the amphoteric water-soluble polymer of the invention is a copolymer of 2-acrylamido-2-methylpropanesulphonic acid and/or salts thereof and methacrylamido propyltrimethyl ammonium chloride, wherein the copolymer comprises a quantity of methacrylamido propyltrimethyl ammonium chloride of at least 30 mol %.

Preferably, the amphoteric water-soluble polymer of the invention is a copolymer of 2-acrylamido-2-methylpropanesulphonic acid and/or salts thereof and methacrylamido propyltrimethyl ammonium chloride, wherein the copolymer has a molar ratio between methacrylamido propyltrimethyl ammonium chloride (MAPTAC) and AMPSA and/or salts thereof between 0.4 and 1.5, preferably between 0.4 and 1.4, preferably between 0.4 and 1.35, more preferably between 0.6 and 1.3.

Preferably, the amphoteric water-soluble polymer of the invention is a copolymer of 2-acrylamido-2-methylpropanesulphonic acid and/or salts thereof and methacrylamido propyltrimethyl ammonium chloride, wherein the copolymer comprises a quantity of methacrylamido propyltrimethyl ammonium chloride of at least 30 mol % and a molar ratio between methacrylamido propyltrimethyl ammonium chloride and AMPSA and/or salts thereof between 0.4 and 1.5, preferably between 0.4 and 1.35, more preferably between 0.6 and 1.3.

Optionally, the amphoteric water-soluble polymer according to the invention may contain C units of acrylamide. The quantity of C units of acrylamide in the amphoteric water-soluble polymer according to the invention is between 0 and 50 mol % relative to the molar quantity of all units included in the polymer. More preferably, it is between 10 and 40 mol %.

Generally, the amphoteric water-soluble polymer according to the invention does not require the development of a particular polymerisation method. Indeed, it can be obtained using all known-art polymerisation techniques. In particular, this may be solution polymerisation, gel polymerisation, precipitation polymerisation, emulsion polymerisation (aqueous or inverse), suspension polymerisation, reactive extrusion polymerisation, water-in-water polymerisation, or micellar polymerisation.

Persons skilled in the art know how to adjust the quantities of monomers used in the polymerisation method to obtain the amphoteric water-soluble polymer according to the invention.

Generally, the polymerisation is free-radical polymerisation, preferably polymerisation in solution. We include within the concept of free-radical polymerisation free-radical polymerization by means of UV, azo, redox, or thermal initiators, as well as controlled radical polymerisation (CRP) techniques or matrix polymerisation techniques.

In one particular embodiment of the invention, amphoteric water-soluble polymer according to the invention has a weight-averaged molecular weight between 100,000 and 5,000,000 g/mol, more preferably between 500,000 and 3,000,000 g/mol.

The weight-averaged molecular weight is determined by high-performance liquid chromatography (HPLC).

Instrument: AGILENT 1260 INFINITY SYSTEM, Wyatt Technology detector

Columns: Shodex SB 807-G

Shodex SB 807-HQ

Shodex SB 805 custom

Method:

Temperature: 25° C.

Mobile phase: 0.4M NaNO₃+100 ppm NaN₃

Injection: 100 μL

Flow: 0.5 ml/min

Detection: Dawn HELEOS (MALS), Optilab T-Rex (RI).

During the analysis to determine the weight-average molecular weight, the sample is prepared at 3000 ppm, filtered at 1.2, then injected twice.

Preferably, the amphoteric water-soluble polymer according to the invention does not comprise branching or crosslinking agents. However, it is possible to use these agents so as to maintain the water-solubility of the polymer.

### Composition According to the Invention

This invention also concerns a composition comprising at least one amphoteric water-soluble polymer, which comprises:
A units of a cationic monomer selected from: acidified or quaternised dimethylaminoethyl acrylate salts, acidified or quaternised dimethylaminoethyl methacrylate salts, acidified or quaternised dimethylaminopropyl acrylamide salts, and acidified or quaternised dimethylaminopropyl methacrylamide salts, and mixtures thereof;
B units of 2-acrylamido-2-methylpropanesulphonic acid (ATBS) and/or salts thereof;
optionally C units of acrylamide,

characterised in that the molar ratio between the A units of cationic monomer and the B units of 2-acrylamido-2-methylpropanesulphonic acid and/or salts thereof is between 0.4 and 1.5.

Preferably, the molar ratio between the A units of cationic monomer and the B units of 2-acrylamido-2-methylpropanesulphonic acid is between 0.4 and 1.4, preferably between 0.4 and 1.35, more preferably between 0.6 and 1.3.

Preferably, the composition according to the invention is a haircare and/or skincare composition.

Advantageously, the composition according to the invention is a transparent haircare and/or skincare composition.

The transparence of the gels obtained is determined by light transmittance measurement on a Hach Lange DR 6000 spectrophotometer. These measurements were carried out at 410 nm in 1.5 mL polystyrene containers.

‘Transparent composition’ refers to a composition having a transmittance value greater than or equal to 80%, preferably greater than or equal to 82%, more preferably greater than or equal to 85%, more preferably greater than or equal to 87%, advantageously greater than or equal to 90%.

Preferably, the composition according to the invention is a detergent composition.

Preferably, the composition according to the invention is a household fabric softener composition.

Preferably, the amount of amphoteric water-soluble polymer in the composition is between 0.01 and 3% of the total mass of the composition.

Preferably, during the production of the composition, the amphoteric water-soluble polymer is first inserted into water and entirely dissolved before the other components are added.

Preferably, the composition according to this invention further comprises:
at least one first surfactant;
at least one second surfactant of a different nature to the first surfactant;
optionally at least one silicone agent;
optionally at least one active agent; and
water.

The composition according to the invention preferably comprises at least two surfactants, who give the composition a cleansing or foaming character. These surfactants are selected from nonionic, anionic, cationic, amphoteric, and zwitterionic surfactants.

Preferably, the first surfactant is anionic, whilst the second surfactant is zwitterionic.

The first anionic surfactant that can be used in the invention is selected from carboxylates, acylamino acides, amodoethercarboxylates, alkyl polyaminocarboxylates, alkyl ether sulphates, alkyl sulphonates, isethionates, alkyl methyltaurates, alkyl sulphosuccinates, alkyl sulphoacetates, alkyl phosphates (mono- or dialkyl phosphates), and salts thereof.

Examples of the first anionic surfactant include sodium lauryl ether sulphate, lauryl glycol carboxylate, cocoampho(di)acetate, lauroampho(di)acetate, and potassium lauryl phosphate. Preferably, the anionic surfactant is sodium lauryl ether sulphate.

Preferably, the quantity of the first anionic surfactant in the composition according to the invention is between 5 and 20% of the total weight of the composition.

The second zwitterionic surfactant that may be used in the invention is selected from betaine derivatives, including am idopropylbetaines, amphoacetates and amphodiacetates, and hydroxylsultaines.

Examples of betaine derivatives include cocoyl betaine, lauroyl betaine, cocamidopropyl betaine, and lauramido propylbetaine. Preferably, the second zwitterionic surfactant is cocamidopropyl betaine.

Preferably, the quantity of the second zwitterionic surfactant in the composition according to the invention is between 0.1 and 10% of the total weight of the composition.

The composition according to the invention, preferably for haircare and/or skincare, preferably comprises at least one silicone agent. Silicone agents are commonly used in haircare compositions because they give softness and shine to hair. They are particles that deposit all around the hair in order to create a protective layer. Silicone agents used for haircare products are versatile ingredients that adapt to different care products and the needs of the hair. They exist in the form of chains, nets, or rings. They may by water-soluble as well as hydrophobic.

Silicone agents that may be used in accordance with the invention may be in the form of oils, waxes, resins, or rubbers.

The silicone agent(s) that may be used in the composition according to the invention, which is preferably for haircare and/or skncare, may be selected from the group comprising polydialkylsiloxanes, in particular polydimethylsiloxanes (PDMS), and organomodified polysiloxanes including at least one moiety selected from amino groups, aryl groups, and alkoxy groups.

Silicones are defined in greater detail in NOLL, Chemistry and Technology of Silicones (1968), Academie Press.

Preferably the quantity of silicone agent in the composition is between 0 and 10% of the total weight of the composition.

The composition according to the invention, which is preferably for haircare and/or skincare, may further contain one or more active agents conventionally used in care products.

Within the meaning of the invention, ‘active agent’ refers to a compound having properties of interest (care, softening, detangling, etc.) that is conventionally used by persons skilled in the art.

Examples include water-soluble and fat-soluble active agents that are conventional in the cosmetic field, such as preservatives, sequestrants (EDTA), antioxidants, fragrances, colourings, soluble colourings or pigments, encapsulated or otherwise, mother-of-pearl, matifying agents, tensioners, lighteners or exfoliants, solar filters, hydrophilic or lipophilic cosmetic or dermatological active agents such as water- or fat-soluble vitamins, antiseptics, anti-seborrhoeic agents, antimicrobials such as benzoyl peroxide, salicylic acid, triclosan, azelaic acid, niacin (vitamin PP), slimming agents such as caffeine, optical brighteners, electrolytes, agents that serve to improve the cosmetic properties of the skin, or solid particles, spherical or otherwise, porous or otherwise, of all sizes.

The quantities of these various active agents are those conventionally used in the field in question, and represent, for example, 0-20% of the total weight of the composition. These agents, as well as their concentrations, must be such that they do not modify the desired properties of the composition according to the invention.

The composition according to the invention, preferably for haircare and/or skincare, preferably comprises water as a preparation solvent. In general, the amount of water in the composition is between 50 and 95% of the total weight of the haircare and/or skincare composition, preferably between 60 and 90% of the total weight of the haircare and/or skincare composition, even more preferably between 70 and 85%.

In addition to water, the composition according to the invention, which is preferably for haircare and/or skincare, may comprise a mixture of water and one or more water-soluble solvents selected from the lower alcohols (mono-ols) having 1-8 carbon atoms, such as ethanol, isopropanol, tert-butanol, n-butanol; and polyols such as glycerine, propylene glycol, butylene glycol, isoprene glycol, glycols such as PEG-8; sorbitol; polyol ethers; sugars such as glucose, fructose, maltose, lactose, sucrose, to the extent that these compounds do not alter the desired properties of the composition according to the invention.

The quantity of this/these solvent(s) in the composition of the invention may range, for example, from 0 to 30 wt %, preferably from 1 to 10 wt % relative to the total weight of the composition.

The quantity of each component of the composition may be adjusted by persons skilled in the art so as not to exceed 100 mass % when prepared according to the invention.

The composition according to the invention, which is preferably for haircare and/or skincare, may take various forms, in particular those of thickened solutions, gels, milks, or creams with varying degrees of thickness. Preferably, the composition according to the invention, which is preferably for haircare and/or skincare, is in the form of thickened solutions or gels.

The composition according to the invention is manufactured using techniques known to persons skilled in the art, who will also know how to use the amphoteric water-soluble polymer according to the invention and the other ingredients in order to obtain a conventional composition.

The haircare and/or skincare composition according to the invention may be used in the form of shampoos, aftershave, sunscreen, hand lotion, liquid soap, bar soap, bath oil bars, shaving cream, conditioners, hair dye, perms, straighteners, hair bleaches, styling products, styling gels, or shower gels. Preferably, the haircare and/or skincare composition according to the invention is used in a shampoo or haircare product.

### Uses and Advantages of the Invention

The composition according to the invention provides good deposition properties due to the presence of a highly cationic polymer. The improved active agent deposition allows for a reduction in the amount of silicone agent used in the haircare and/or skincare composition compared to prior-art cosmetic haircare and/or skincare compositions. The reduction in the amount of silicone agent also allows for a reduced environmental impact, as such agents are commonly harmful to the environment.

All of these advantages combine with the production of a transparent compound, as desired by the formulators of cosmetic haircare and/or skincare products.

The amphoteric water-soluble polymer according to the invention may also be used to improve active agent deposition in a detergent or household fabric softener compositions, as these polymers have greater affinity to all types of negatively charged surfaces.

The invention and its benefits will be made clearer by the following examples, which are provided by way of example only and without limitation.

### FIGURES

FIG. 1 is a graph showing the development of coacervation as a function of the dilution rate of the polymer of compositions 21-30.

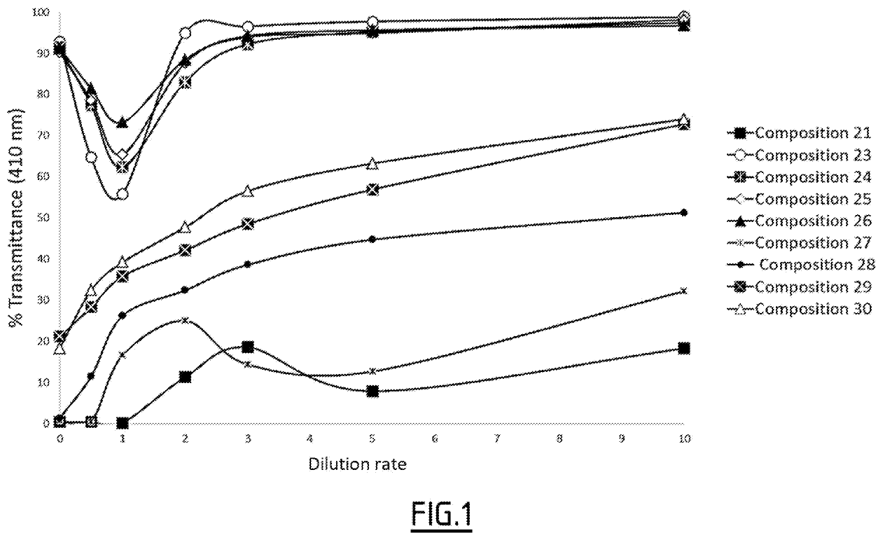

FIG. 2 is a graph showing the development of coacervation as a function of the dilution rate of the polymer of compositions 32-40.

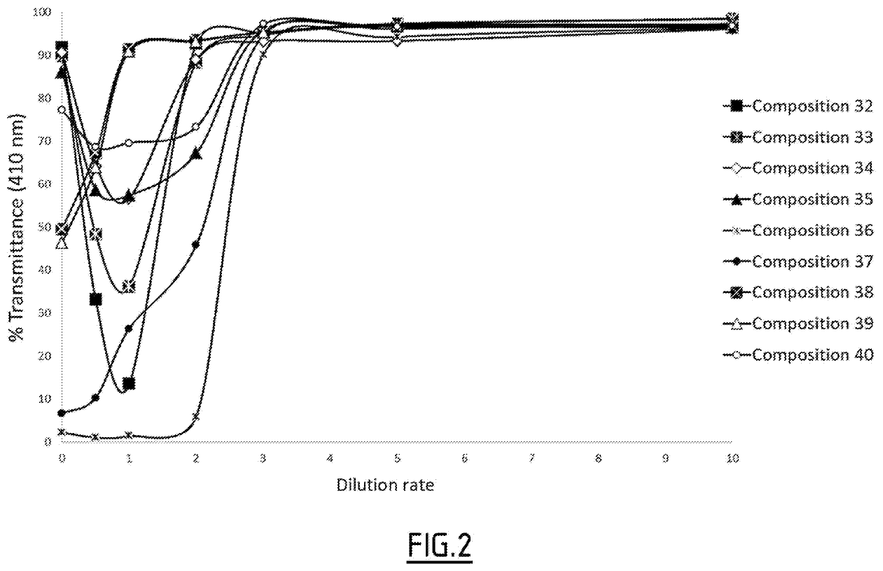

### EXAMPLES

### 1. Polymer Synthesis

Polymer 1: 245.2 g water, 102.8 g 50 acrylamide, followed by 318.5 g 50 MAPTAC were inserted into a jacketed reactor with mechanical stirring with a semicircular blade. The mixture was stirred at 50° C., and the pH adjusted to a value of 5.0 with citric acid, and then degassed for 30 min. 0.6 g sodium persulphate and 0.01 g sodium metabisulphite were added in order to initiate the reaction. When the temperature increased by 2° C., the degassing was ended, and the temperature allowed to rise without additional heating (exothermic reaction). Once the temperature was stabilised, the solution was left to stir for 1 h. The mixture was then diluted with 309 g water, then 2 g sodium metabisulphite was added. After homogenisation, sodium benzoate was added; once dissolved, the pH was adjusted to between 4.0 and 4.5 with citric acid. Thus, an aqueous polymer solution was obtained.

Other polymers were also obtained from various monomers (acrylamido propyltrimethyl ammonium chloride (APTAC), quaternised dimethylaminoethyl acrylate, quaternised dimethylaminoethyl methacrylate (DMAEMA), N-dimethylamino-3-propyl-1-methacrylamide (DMAPMA), 2-acrylamido-2-methylpropanesulphonic acid (AMPSA) and/or salts thereof, acrylamide (AM) or acrylic acid (AA) and/or salts thereof) following the same protocol.

The compositions of the polymers obtained are set forth in table 1.

TABLE 1
Copolymer composition

| | Molar | | | | | | | | |
|---|---|---|---|---|---|---|---|---|---|
| | ratio | Composition of copolymers (mol %) | | | | | | | |
| | C+/A− | MAPTAC | APTAC | DMAEMA | DMAEA | ATBS | DMAPMA | AA | AM |
| Polymer 1 | 1 | 50 | — | — | — | — | — | — | 50 |
| (Comparative) | | | | | | | | | |
| Polymer 2 | 1 | 50 | — | — | — | — | — | 50 | — |
| (Comparative) | | | | | | | | | |
| Polymer 3 | 1 | 50 | — | — | — | 50 | — | — | — |
| (invention) | | | | | | | | | |
| Polymer 4 | 1 | — | 50 | — | — | 50 | — | — | — |
| (invention) | | | | | | | | | |
| Polymer 5 | 1 | — | — | 50 | — | 50 | — | — | — |
| (invention) | | | | | | | | | |
| Polymer 6 | 1 | — | — | — | 50 | 50 | — | — | — |
| (invention) | | | | | | | | | |
| Polymer 1′ | 1.3 | 56.5 | — | — | — | 43.5 | — | — | — |
| (invention) | | | | | | | | | |
| Polymer 2′ | 1.3 | — | 56.5 | — | — | 43.5 | — | — | — |
| (invention) | | | | | | | | | |
| Polymer 3′ | 1.3 | — | — | 56.5 | — | 43.5 | — | — | — |
| (invention) | | | | | | | | | |
| Polymer 4′ | 1.3 | — | — | — | 56.5 | 43.5 | — | — | — |
| (invention) | | | | | | | | | |
| Polymer 7 | 1.85 | 65 | — | — | — | 35 | — | — | — |
| (Comparative) | | | | | | | | | |
| Polymer 8 | 1.85 | — | 65 | — | — | 35 | — | — | — |
| (Comparative) | | | | | | | | | |
| Polymer 9 | 1.85 | — | — | 65 | — | 35 | — | — | — |
| (Comparative) | | | | | | | | | |
| Polymer 10 | 1.85 | — | — | — | 65 | 35 | — | — | — |
| (Comparative) | | | | | | | | | |
| Polymer 5′ | 1.5 | 60 | — | — | — | 40 | — | — | — |
| (Comparative) | | | | | | | | | |
| Polymer 6′ | 1.5 | — | 60 | — | — | 40 | — | — | — |
| (Comparative) | | | | | | | | | |
| Polymer 7′ | 1.5 | — | — | 60 | — | 40 | — | — | — |
| (Comparative) | | | | | | | | | |
| Polymer 8′ | 1.5 | — | — | — | 60 | 40 | — | — | — |
| (Comparative) | | | | | | | | | |
| Polymer 9′ | 1.5 | 5 | | | | 10 | 10 | 75 | — |
| (Comparative) | | | | | | | | | |

| | | |
|---|---|---|
| Polymer 20 | — | Cationic guar |
| (reference) | | |

TABLE 2
Terpolymer composition

| | Molar | | | | | | | |
|---|---|---|---|---|---|---|---|---|
| | ratio | Composition of terpolymers (mol %) | | | | | | |
| | C+/A− | MAPTAC | APTAC | DMAEMA | DMAEA | ATBS | AA | AM |
| Polymer 11 | 1.14 | 40 | — | — | — | — | 35 | 25 |
| (Comparative) | | | | | | | | |
| Polymer 12 | 1.14 | 40 | — | — | — | 35 | — | 25 |
| (invention) | | | | | | | | |
| Polymer 13 | 1.14 | — | 40 | — | — | 35 | — | 25 |
| (invention) | | | | | | | | |
| Polymer 14 | 1.14 | — | — | 40 | — | 35 | — | 25 |
| (invention) | | | | | | | | |
| Polymer 15 | 1.14 | — | — | — | 40 | 35 | — | 25 |
| (invention) | | | | | | | | |
| Polymer 16 | 2 | 40 | — | — | — | 20 | — | 40 |
| (Comparative) | | | | | | | | |
| Polymer 17 | 2 | — | 40 | — | — | 20 | — | 40 |
| (Comparative) | | | | | | | | |
| Polymer 18 | 2 | — | — | 40 | — | 20 | — | 40 |
| (Comparative) | | | | | | | | |
| Polymer 19 | 2 | — | — | — | 40 | 20 | — | 40 |
| (Comparative) | | | | | | | | |

| | | |
|---|---|---|
| Polymer 20 | — | Cationic guar |
| (reference) | | |

### 2. Production of Transparent Compositions for Skincare and/or Haircare

The compositions were prepared from 0.3 mass % of the polymers prepared previously. Polymer 1 was used for the preparation of composition 1, polymer 2 for composition 2, etc.

Each polymer (0.3 mass %) was added to water stirred at 300 rpm until complete homogenisation. With continuing stirring, sodium lauryl ether sulphate (SLES), followed by cocoamidopropyl betaine (CAPB),and lastly sodium benzoate were added. Once the sodium benzoate was completely dissolved, the pH of the composition was adjusted to 4.8 with citric acid. Once the pH was fixed at 4.8, NaCl was added in order to thicken the medium. Thus, analysable gels were obtained.

TABLE 3
Shampoo composition

| INGREDIENT | MASS % |
|---|---|
| SLES | 10.0 |
| CAPB | 2.5 |
| Polymer | 0.3 |
| Sodium Benzoate | 0.5 |
| Citric acid | QSP pH 4.8 |
| NaCl | 1.5 |
| Water | QSP 100% |

QSP 100% = quantity of water sufficient to reach 100%
QSP pH 4.8 = quantity sufficient to reach pH 4.8.

### 3. Measuring the Transparence of the Compositions in Gel Form

The transparence of was gels obtained is determined by light transmittance measurement on a Hach Lange DR 6000 spectrophotometer. These measurements were carried out at 410 nm in 1.5 mL polystyrene containers.

In the context of the invention, the applicant deems a composition to be transparent if it has a measured transmittance value greater than 80% T at 410 nm.

The polymers were tested in the compounds previously produced.

The results are shown in tables 4 and 5.

TABLE 4
Results of transmittance measurements of
compositions 1-10 and 1′-9′

| | Transmittance measurement |
|---|---|
| | (% T, 410 nm) |
| Composition 1 (Comparative) | 0.4 |
| Composition 2 (Comparative) | NA (dephasing) |
| Composition 3 (Invention) | 92.8 |
| Composition 4 (Invention) | 91.4 |
| Composition 5 (Invention) | 90.2 |
| Composition 6 (Invention) | 91.2 |
| Composition 1′ (invention) | 86.1 |
| Composition 2′ (invention) | 85.2 |
| Composition 3′ (invention) | 85.7 |
| Composition 4′ (invention) | 85.9 |
| Composition 7 (Comparative) | 0.8 |
| Composition 8 (Comparative) | 1.4 |
| Composition 9 (Comparative) | 21.2 |
| Composition 10 (Comparative) | 18.4 |
| Composition 5′ (Comparative) | 10.2 |
| Composition 6′ (Comparative) | 8.2 |
| Composition 7′ (Comparative) | 2.3 |
| Composition 8′ (Comparative) | 14.9 |
| Composition 9′ (Comparative) | 20.8 |

Only the compositions containing the copolymer according to the invention (compositions 3-6 and 1′-4′) are transparent, with a transmittance greater than 80% T.

The composition produced with a copolymer comprising acrylic acid in lieu of ATBS is dephased, rendering measurement impossible (composition 2).

Where the proportions of cationic monomers was not satisfactory (mol % of cationic monomer greater than 60 or molar ratio not between 0.4 and 1.4), the compositions were opaque (compositions 1, 7-10, and 5′-9′). In particular, a tetrapolymer such as that described in EP 0 139 588 (polymer 9′) does not result in a transparent composition.

TABLE 5
Results of transmittance measurements for compositions 11-20

| | Transmittance measurement |
|---|---|
| | (% T, 410 nm) |
| Composition 11 (Comparative) | NA (dephasing) |
| Composition 12 (Invention) | 91.8 |
| Composition 13 (Invention) | 89.8 |
| Composition 14 (Invention) | 90.5 |
| Composition 16 (Comparative) | 2.3 |
| Composition 17 (Comparative) | 6.7 |
| Composition 18 (Comparative) | 49.6 |
| Composition 19 (Comparative) | 46.4 |
| Composition 20 (Reference) | 77.2 |

Only the compositions containing the copolymer according to the invention (compositions 12-15) are transparent, with a transmittance greater than 80% T.

Replacing ATBS with acrylic acid dephased the composition, rendering measurement impossible (composition 11).

Where the proportions of cationic monomers was not satisfactory (mol % of cationic monomer greater than 60 or molar ratio not between 0.4 and 1.5), the compositions were opaque (compositions 16-19).

As for the reference composition (cationic guar), the composition was cloudy and did not provide a satisfactory result.

### 4. Active Agent Deposition Determination

To determine the efficacy of the aid to deposition of the amphoteric water-soluble polymers according to the invention, 7.5 mass % of a silicone agent emulsion (Xiameter MEM 8177 from Dow Chemical) was added at the end of the process to compositions 1-20 and 1′-9′, described supra, in order to obtain compositions 21-40 and 21′-29′, respectively.

The light transmittance of compositions 21-40 and 21′-29′ was measured on a Hach Lange DR 6000 spectrophotometer at a wavelength of 410 nm in 1.5 mL polystyrene containers.

The efficacy of the deposition aid was determined using coacervation tests that simulate hair washing and comprise the following steps:
the composition must be transparent at the outset, indicating a transparent composition in the shampoo bottle, etc.;
then, during dilutions with water, representing the dilution of the compound on wet hair, the compound must become cloudy (coacervate formation), thus indicating the deposition of active agent on the hair;
then, successive dilutions simulate the rinsing of the hair; the composition must again become transparent.

Coacervate formation during dilution is shown by a reduction in transmittance (appearance of cloudiness).

Table 6 summarises the transmittance results for compositions 21-30 and 21′-29 during the coacervation tests.

TABLE 6
Copolymer coacervation test

| | Copolymer dilution | | | | | | |
|---|---|---|---|---|---|---|---|
| | 0 | 0.5 | 1 | 2 | 3 | 5 | 10 |
| Composition 21 | 0.4 | 0.4 | 0.2 | 11.3 | 18.6 | 7.9 | 18.3 |
| (Comparative) | | | | | | | |
| Composition 22 | NA (dephasing) | | | | | | |
| (Comparative) | | | | | | | |
| Composition 23 | 92.8 | 64.7 | 55.8 | 94.9 | 96.5 | 97.7 | 98.8 |
| (Invention) | | | | | | | |
| Composition 24 | 91.4 | 77.4 | 62.3 | 83.0 | 92.2 | 95.2 | 97.4 |
| (Invention) | | | | | | | |
| Composition 25 | 90.2 | 78.5 | 65.4 | 87.6 | 93.9 | 94.9 | 98.1 |
| (Invention) | | | | | | | |
| Composition 26 | 91.2 | 81.4 | 73.2 | 88.4 | 94.2 | 95.6 | 96.7 |
| (Invention) | | | | | | | |
| Composition 27 | 0.8 | 0.5 | 16.6 | 25 | 14.4 | 12.7 | 32.2 |
| (Comparative) | | | | | | | |
| Composition 28 | 1.4 | 11.5 | 26.2 | 32.4 | 38.6 | 44.7 | 51.3 |
| (Comparative) | | | | | | | |
| Composition 29 | 21.2 | 28.3 | 35.8 | 42.1 | 48.5 | 56.9 | 72.8 |
| (Comparative) | | | | | | | |
| Composition 30 | 18.4 | 32.4 | 39.2 | 47.7 | 56.5 | 63.2 | 74.0 |
| (Comparative) | | | | | | | |
| Composition 21′ | 86.1 | 58.7 | 47.4 | 67.2 | 95.8 | 96.1 | 96.4 |
| (invention) | | | | | | | |
| Composition 22′ | 85.2 | 60.1 | 48.6 | 72.3 | 92.6 | 95.6 | 96.1 |
| (invention) | | | | | | | |
| Composition 23′ | 85.7 | 59.3 | 48.3 | 70.1 | 94.9 | 95.2 | 95.4 |
| (invention) | | | | | | | |
| Composition 24′ | 85.9 | 61.7 | 51.1 | 69.7 | 93.6 | 95.4 | 95.9 |
| (invention) | | | | | | | |
| Composition 25′ | 10.2 | 4.4 | 2.4 | 6.1 | 50.4 | 96.9 | 97.6 |
| (Comparative) | | | | | | | |
| Composition 26′ | 8.2 | 12.7 | 25 | 32.9 | 50.8 | 77.3 | 80.4 |
| (Comparative) | | | | | | | |
| Composition 27′ | 2.3 | 4.9 | 18.9 | 44.6 | 78.1 | 80.1 | 84.2 |
| (Comparative) | | | | | | | |
| Composition 28′ | 14.9 | 16.7 | 21.4 | 31.4 | 56.8 | 77.4 | 88.9 |
| (Comparative) | | | | | | | |
| Composition 29′ | 20.8 | 25 | 29 | 36 | 41.4 | 55.8 | 74 |
| (Comparative) | | | | | | | |

Compositions 21 and 22, which were made without ATBS or with acrylic acid in lieu of ATBS, were not satisfactory. Composition 1 was not clear. As for composition 2, it dephased and did not allow for comparison tests to be conducted.

Compositions that did not comply with the cationic monomer/ATBS molar ratio according to the invention did not perform well, both in terms of poor transparence at the start (compositions 27-30 and 25′-29′), or poor elimination during simulated rinsing (compositions 27-28).

Only the compositions comprising amphoteric water-soluble polymers according to the invention (compositions 23-26 and 21′-24′) offered the expected results:
transparence in the initial state, and then following 0.5-1 dilutions;
   coacervate formation, indicating active agent deposition and with continued dilution;
good elimination, returning to a transparent solution.

Table 7 summarises the transmittance results for compositions 31-40 during the coacervation tests.

TABLE 7
Polymer coacervation test

| | Terpolymer dilution | | | | | | |
|---|---|---|---|---|---|---|---|
| | 0 | 0.5 | 1 | 2 | 3 | 5 | 10 |
| Composition 31 | NA (dephasing) | | | | | | |
| (Comparative) | | | | | | | |
| Composition 32 | 91.8 | 33.2 | 13.6 | 92.7 | 95.2 | 97.3 | 98.5 |
| (Invention) | | | | | | | |
| Composition 33 | 89.8 | 48.3 | 36.2 | 88.3 | 94.6 | 97.1 | 98.4 |
| (Invention) | | | | | | | |
| Composition 34 | 90.5 | 64.3 | 56.7 | 88.9 | 93.2 | 93.3 | 96.1 |
| (Invention) | | | | | | | |
| Composition 35 | 86.1 | 58.7 | 47.4 | 67.2 | 95.8 | 96.6 | 96.4 |
| (Invention) | | | | | | | |
| Composition 36 | 2.3 | 1.1 | 1.5 | 6.0 | 90.1 | 94.2 | 96.4 |
| (Comparative) | | | | | | | |
| Composition 37 | 6.7 | 10.4 | 26.4 | 45.9 | 94.6 | 96.1 | 97.2 |
| (Comparative) | | | | | | | |
| Composition 38 | 49.6 | 67.3 | 91.4 | 93.4 | 95.2 | 96.8 | 96.7 |
| (Comparative) | | | | | | | |
| Composition 39 | 46.4 | 64.0 | 90.8 | 93.2 | 95.3 | 97.0 | 97.2 |
| (Comparative) | | | | | | | |
| Composition 40 | 77.2 | 68.6 | 69.6 | 73.3 | 97.2 | 96.6 | 96.8 |
| (Reference) | | | | | | | |

Composition 31, which was made with acrylic acid in lieu of ATBS, dephased and did not allow comparison tests to be conducted.

Compositions that did not comply with the cationic monomer/ATBS molar ratio according to the invention did not perform well, both in terms of poor transparence at the start (compositions 36-39), or poor elimination during simulated rinsing (compositions 38-39).

Only the compositions comprising amphoteric water-soluble polymers according to the invention (compositions 32-35) offered the expected results:
transparence in the initial state, and then following 0.5-1 dilutions;
coacervate formation, indicating active agent deposition and with continued dilution;
good elimination, returning to a transparent solution.

Guar (composition 40) offered average performance that was not satisfactory in terms of active agent deposition and transparence.

1. Composition comprising at least one water-soluble amphoteric polymer, which comprises:
A units of a cationic monomer selected from the group consisting of acidified or quaternised dimethylaminoethyl acrylate salts, acidified or quaternised dimethylaminoethyl methacrylate salts, acidified or quaternised dimethylaminopropyl acrylamide salts, and acidified or quaternised dimethylaminopropyl methacrylamide salts, and mixtures thereof;
B units of 2-acrylamido-2-methylpropanesulphonic acid and/or salts thereof; and
optionally C units of acrylamide,
wherein the molar ratio between the A units of cationic monomer and the B units of 2-acrylamido-2-methylpropanesulphonic acid and/or salts thereof is between 0.4 and 1.4.

2. Composition according to claim 1, wherein the quantity of A units of cationic monomer in the water-soluble amphoteric polymer is between 30 and 60 mol % relative to the molar quantity of all units included in the polymer.

3. Composition according to claim 1, wherein the quantity of B units of 2-acrylamido-2-methylpropanesulphonic acid in the water-soluble amphoteric polymer is between 20 and 70 mol % relative to the molar quantity of all units included in the polymer.

4. Composition according to claim 1, wherein the quantity of C units of acrylamide in the water-soluble amphoteric polymer is between 0 and 50 mol % relative to the molar quantity of all units included in the polymer.

5. Composition according to claim 1, wherein the weight-average molecular weight of the water-soluble amphoteric polymer is between 100,000 and 5,000,000 g/mol.

6. Composition according to claim 1, wherein the water-soluble amphoteric polymer is a copolymer of methacrylamidopropyltrimethyl ammonium chloride and 2-acrylamido-2-methylpropanesulphonic acid and/or salts thereof.

7. Composition according to claim 1, wherein the quantity of water-soluble amphoteric polymer is between 0.01 and 3% of the total mass of the composition.

8. Composition according to claim 1, further comprising:
at least one first surfactant;
at least one second surfactant of a different nature to the first surfactant;
optionally at least one silicone agent;
optionally at least one active agent; and
water.

9. Composition according to claim 1, wherein the molar ratio of the A units of cationic monomer and the B units of 2-acrylamido-2-methylpropanesulphonic acid and/or salts thereof is between 0.4 and 1.35.

10. Composition according to claim 1, wherein it is a haircare and/or skincare composition.

11. Composition according to claim 1, wherein it is a detergent composition.

12. Composition according to claim 1, wherein it is a household fabric softener composition.

13. Amphoteric water-soluble polymer, comprising:
A units of a cationic monomer selected from the group consisting of acidified or quaternised dimethylaminoethyl acrylate salts, acidified or quaternised dimethylaminoethyl methacrylate salts, acidified or quaternised dimethylaminopropyl acrylamide salts, and acidified or quaternised dimethylaminopropyl methacrylamide salts, and mixtures thereof;
B units of 2-acrylamido-2-methylpropanesulphonic acid and/or salts thereof; and
optionally C units of acrylamide,
wherein the molar ratio between the A units of cationic monomer and the B units of 2-acrylamido-2-methylpropanesulphonic acid and/or salts thereof is between 0.4 and 1.4.

14. Method to improve the aid to the deposition of an active agent in a haircare and/or skincare composition, comprising the introduction of an amphoteric water-soluble polymer according to claim 13 in said composition.

15. Method to improve the aid to the deposition of an active agent in a detergent composition, comprising the introduction of an amphoteric water-soluble polymer according to claim 13 in said composition.

16. Method to improve the aid to the deposition of an active agent in a household fabric softener composition, comprising the introduction of an amphoteric water-soluble polymer according to claim 13 in said composition.
